(19) United States
(12) Patent Application Publication
(10) Pub. No.: US 2021/0016100 A1
(43) Pub. Date: Jan. 21, 2021

(54) Coated Implantable Medical Device and Coating Method

(71) Applicant: BIOTRONIK SE & Co. KG, Berlin (DE)

(72) Inventors: Matthias FROTSCHER, Rostock (DE); Marco BOSSELMANN, Rostock (DE); Carsten MOMMA, Rostock (DE); Jeffrey A. VON ARX, Lake Oswego, OR (US); Wantjinarjo SUWITO, West Linn, OR (US); Dirk MUESSIG, West Linn, OR (US); Hannes KRAETSCHMER, West Linn, OR (US)

(73) Assignee: BIOTRONIK SE & Co. KG, Berlin (DE)

(21) Appl. No.: 17/043,269

(22) PCT Filed: Mar. 15, 2019
(86) PCT No.: PCT/EP2019/056523

§ 371 Date: Sep. 29, 2020

(30) Foreign Application Priority Data
Apr. 4, 2018 (EP) 18165615.8

(51) Int. Cl. A61N 1/375; A61N 1/05; A61L 31/02; A61L 31/08; A61L 31/10

(52) U.S. Cl. CPC A61N 1/37518; A61N 1/0573; A61N 1/37512; A61L 31/022; A61L 2400/16; A61L 31/088; A61L 31/10; A61L 2420/02; A61L 31/084

(57) ABSTRACT

An intracardiac pacing system comprising a fixation element for fixing the intracardiac pacing system to body tissue is disclosed. The fixation element comprises a metallic material, and is at least partially coated with a metal-ion release inhibiting material. Also, a method for coating at least part of an intracardiac pacing system is disclosed.

### CROSS-REFERENCE TO RELATED APPLICATIONS

This application is the United States national phase under 35 U.S.C. § 371 of PCT International Patent Application No. PCT/EP2019/056523, filed on Mar. 15, 2019, which claims the benefit of European Patent Application No. 18165615.8, filed on Apr. 4, 2018 the disclosures of which are hereby incorporated by reference herein in their entireties.

### TECHNICAL FIELD

The present disclosure relates to a coated implantable medical device and coating method.

### BACKGROUND

When a medical device is implanted in a patient, biocompatibility is always an issue. In particular, metal ions released from the device into body tissue of the patient may cause an inflammatory reaction which may interfere with the healing process.

U.S. Publication No. 2013/0253345 discloses an implantable medical device with a set of fixation tines. The tines are made of nitinol and are coated with a radiopaque coating, a tissue growth promoter or a tissue growth inhibitor.

U.S. Pat. No. 5,238,866 relates to an amorphous semi-conductive surface coating for a cardiovascular implant in order to reduce the risk of valve occlusions by thrombus, thromboembolism and anti-coagulant-related hemorrhage.

European Publication No. EP 0 371 908 A1 discloses an implant with a semiconducting coating, for example amorphous silicon carbide, in order to form an antithrombotic surface.

U.S. Publication No. 2010/0114284 discloses an electrode line which is partially coated with an antithrombotic silicon carbide coating.

The present disclosure is directed toward overcoming one or more of the above-mentioned problems, though not necessarily limited to embodiments that do.

### SUMMARY

It is an object to provide improved technologies for implantable devices. In particular, tolerability of the implantable device shall be improved.

An implantable medical device according to claim 1 and a method according to claim 10 are provided. Further embodiments are subject matter of dependent claims.

In one aspect, an implantable medical device which is at least partially coated with a metal-ion release inhibiting material is provided. The implantable medical device may be an intracardiac pacing system. The implantable medical device may comprise a fixation element for fixing the implantable medical device to body tissue. The fixation element may comprise a metallic material. The fixation element may be at least partially coated with the metal-ion release inhibiting material. The fixation element may be arranged on a housing, e.g. at a distal end of the housing. The intracardiac pacing system may be implanted in the heart, e.g. in the ventricle or in the atrium. In this case, the fixation element anchors the intracardiac pacing system in heart tissue, e.g. the myocardium.

A “metal-ion release inhibiting material” is a material that reduces the released ion concentration from a surface coated with the material to 50% or less compared to the released ion concentration from an un-coated surface. Depending on the atom radii of the ion in question, a reduction of ion release of more than 90% can be reached. Hereby, a transfer of metal ions from the medical device (e.g. from the fixation element) to body tissue is reduced or completely inhibited. Ion release can be tested with the ASTM F3306-19 standard test method for ion release evaluation of medical implants. ASTM International (formerly known as American Society for Testing and Materials) is an international standards organization that develops and publishes voluntary consensus technical standards for a wide range of materials, products, systems, and services.

In another aspect, a method for coating an implantable medical device is disclosed. The method comprises steps of providing the implantable medical device, and depositing a metal-ion release inhibiting material on at least a portion of the device. The method may further comprise steps of: providing a fixation element for fixing the implantable medical device to body tissue, wherein the fixation element comprises a metallic material, and depositing a metal-ion release inhibiting material on at least a portion of the fixation element.

The implantable medical device may comprise a housing. The housing may comprise a metallic material. The housing may be at least partially coated with the metal-ion release inhibiting material.

The fixation element may be made of a metallic material. The fixation element may be completely coated with the metal-ion release inhibiting material.

In one embodiment, the fixation element may comprise a shape memory alloy, e.g. nitinol. The fixation element may be made of a shape memory alloy, in particular nitinol. Nickel titanium, also known as nitinol, is a metal alloy of nickel and titanium, where the two elements are present in roughly equal atomic percentage.

The metal-ion release inhibiting material may be a Ni-ion release inhibiting material, thus, a material which suppresses release of Ni ions (e.g. from the fixation element). A “Ni-ion release inhibiting material” is a material that reduces the released Ni ion concentration from a surface coated with the material to 90% or less (up to 96% reduction) compared to the released Ni ion concentration from an un-coated surface.

The metal-ion (e.g. Ni-ion) release inhibiting material may be an amorphous material.

The metal-ion release inhibiting material may be selected from the group of: a-SiₓC₁₋ₓ:H (amorphous hydrogenated silicon carbide, in the following called “a-SiC coating”), TiN (titanium nitride), diamond-like carbon, and parylene.

The fixation element may comprise one or more tines. In one embodiment, the fixation element comprises four tines. The tines may be arranged equally distributed at a distal end of the housing. Alternatively, the fixation element may be provided as a screw element.

The metal-ion release inhibiting material may have a thickness of 1-1000 nm, e.g. 80-200 nm.

The intracardiac pacing system may comprise a lead extension with a further fixation element for fixing the lead extension to body tissue, wherein the further fixation element may comprise a metallic material, and wherein the further fixation element may be at least partially coated with the metal-ion release inhibiting material. For the further fixation element and for the metal-ion release inhibiting material of the further fixation element, the embodiments relating to the fixation element and to the coating of the fixation element disclosed herein are also applicable.

In one embodiment, the implantable medical device is an intracardiac pacing system which comprises several tines (also called a tine array) which may protrude from a cap of a header of the intracardiac pacing system. The tines comprise nitinol (or are made of nitinol) and are partially or completely coated with a-SiₓC₁₋ₓ:H. The a-SiC coating reduces the amount of released Ni ions and leads to an improvement of the overall biocompatibility of the intracardiac pacing system.

The features disclosed herein in regard with the device may also apply to the method and vice versa. Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Following, exemplary embodiments are described with reference to figures. Here show:

FIG. 1 a first embodiment of an intracardiac pacing system, and

FIG. 2 a second embodiment of an intracardiac pacing system.

Same reference numerals are used for same components.

### DETAILED DESCRIPTION

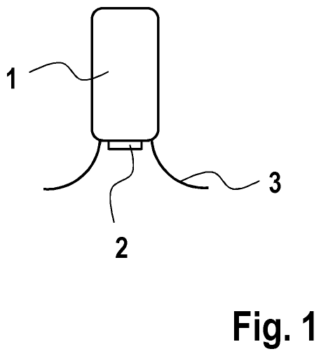

FIG. 1 shows an intracardiac pacing system 1 with a pacing electrode 2 and tines 3. The pacing electrode 2 is disposed on a lower end of the intracardiac pacing system. The tines 3 protrude from the lower end and form a tine array. After implantation of the intracardiac pacing system 1, the system is fixed in the heart (e.g. in the ventricle or in the atrium) by the tines 3 which penetrate heart tissue (e.g. the myocardium).

The tines 3 are used to anchor the intracardiac pacing system in the myocardium and ensure contact of the pacing electrode 2 with the tissue. The tines 3 are in permanent contact with tissue following implantation. The tines 3 are also in close proximity to the pacing electrode 2. Making the tines more biocompatible leads to less body rejection. This may result in a slower endo encapsulation rate, and/or a lower pacing threshold.

The tines 3 are made of nitinol and at least partially coated with a-SiₓC₁₋ₓ:H. In one embodiment, the tines 3 are completely coated with the a-SiC coating. Nitinol is known to elute Ni ions, which may cause harm in the human body. By applying the a-SiC coating on the surface of the tines 3, the amount of released Ni into the tissue and corresponding inflammatory reactions can be significantly reduced. By covering the surface of the tines 3 with the a-SiC coating, the immune system does not react as much to the intracardiac pacing system 1 (being a foreign object in the body) as it may do without the coating. The a-SiC coating has a thickness of 80-200 nm. The nitinol matrix of the base metal will be no longer in contact with body fluids and tissue.

The intracardiac pacing system may comprise a housing. In the housing, a battery and an electronic module may be arranged. The battery is configured to provide electrical energy to the components of the intracardiac pacing system, in particular to the electronic module and to the pacing electrode. The electronic module may comprise a processor and/or memory. The processor may be configured for sensing and/or pacing operation of the intracardiac pacing system. A second electrode (e.g. a ring electrode) may be arranged on the housing, e.g. at a proximal end of the housing.

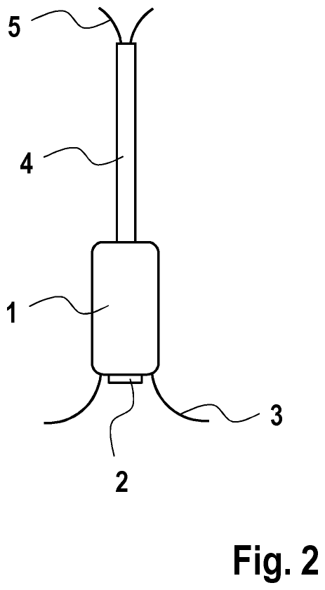

In the embodiment shown in FIG. 2, the intracardiac pacing system 1 comprises a lead extension 4. At a distal end of the lead extension 4, further tines 5 are disposed. The further tines 5 are also made of nitinol and (partially or completely) coated with an a-SiC coating. In this embodiment, the main body of the intracardiac pacing system 1 may be anchored in the ventricle by the tines 3 and the lead extension may be anchored in the atrium by the further tines 5. The intracardiac pacing system 1 may be used in a VDD (ventricular pacing, dual chamber sensing, triggered and inhibited mode) and/or DDD (dual chamber pacing and sensing, triggered and inhibited mode) pacing embodiment.

Following, a procedure for coating the tines 3 and/or the further tines 5 with an a-SiC coating is described. The surface coating of the tines/further tines is produced by way of a PECVD (plasma enhanced chemical vapor deposition) process. As coating material, amorphous hydrogenated silicon carbide (a-SiₓC₁₋ₓ:H) is used. A suitable a-SiC coating can be obtained, if the supplied process gases of the PECVD process are 10% silane (SiH₄) diluted with hydrogen as the silicon supply, and pure methane (CH₄) for carbon supply alloying together with 0.1% of phosphine (PH₃) also diluted with hydrogen. Suitable process parameters are:

| | |
|---|---|
| pressure range: | 0.02-1 mbar; |
| gas composition: | 50-80% methane, 100-0% silane, 0-2% phosphine; |
| gas flow: | 10-50 sccm silane; |
| substrate tempera- | 0-350° C. |
| ture: | |

The features disclosed in the specification, the claims and the figures may be relevant for realizing embodiments either alone or in any combination with each other. It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 intracardiac pacing system

2 pacing electrode

3 tines

4 lead extension

5 further tines

1. An intracardiac pacing system comprising a fixation element for fixing the intracardiac pacing system to body tissue, wherein the fixation element comprises a metallic material, and wherein the fixation element is at least partially coated with a metal-ion release inhibiting material.

2. The system of claim 1, wherein the fixation element comprises a shape memory alloy.

3. The system of claim 2, wherein the fixation element comprises nitinol.

4. The system of claim 1, wherein the metal-ion release inhibiting material is a Ni-ion release inhibiting material.

5. The system of claim 1, wherein the metal-ion release inhibiting material is an amorphous material.

6. The system of claim 1, wherein the metal-ion release inhibiting material is selected from the group of: a-SiₓC₁₋ₓ:H, TiN, diamond-like carbon, and parylene.

7. The system of claim 1, wherein the fixation element comprises one or more tines.

8. The system of claim 1, further comprising a lead extension with a further fixation element for fixing the lead extension to body tissue, wherein the further fixation element comprises a metallic material, and wherein the further fixation element is at least partially coated with the metal-ion release inhibiting material.

9. The system of claim 1, wherein the metal-ion release inhibiting material has a thickness of 1 nm to 1000 nm.

10. A method for coating at least part of an intracardiac pacing system, comprising steps of:
providing the intracardiac pacing system, wherein the intracardiac pacing system comprises a fixation element for fixing the intracardiac pacing system to body tissue, and wherein the fixation element comprises a metallic material, and
depositing a metal-ion release inhibiting material on at least a portion of the fixation element.
